(19) United States
(12) Patent Application Publication
(10) Pub. No.: US 2026/0002855 A1
(43) Pub. Date: Jan. 1, 2026

(54) BIONIC REGULATION METHOD FOR NUMBER OF BOUNCES OF DROPLET ON METAL SURFACE BASED ON ACOUSTIC IN-SITU MONITORING

(71) Applicant: JILIN UNIVERSITY, Changchun (CN)

(72) Inventors: ZHICHAO MA, Changchun (CN); SHENGTENG ZHAO, Changchun (CN); LIBO TAN, Changchun (CN); JIAKAI LI, Changchun (CN); CHAOFAN LI, Changchun (CN); HONGWEI ZHAO, Changchun (CN); LUQUAN REN, Changchun (CN)

(21) Appl. No.: 18/769,988

(22) Filed: Jul. 11, 2024

(30) Foreign Application Priority Data
Jun. 26, 2024 (CN) 202410840471.7

(51) Int. Cl. G01N 13/02; B05D 5/08; B05D 7/14; G01N 33/208

(52) U.S. Cl. CPC G01N 13/02; B05D 5/08; B05D 7/14; G01N 33/208; G01N 2013/0258

(57) ABSTRACT

The present invention discloses a bionic regulation method for a number of bounces of a droplet on a metal surface based on acoustic in-situ monitoring, which comprises the following steps: obtaining dynamic contact characteristics and acoustic properties of a solid-liquid interface of metal superhydrophobic surfaces with different surface information in the droplet bounce process based on an optical-acoustic synchronous in-situ testing system for droplet bounces; and obtaining a correlation among the metal superhydrophobic surface, the acoustic response and the droplet bounce behavior based on the dynamic contact characteristics and the acoustic properties of the solid-liquid interface, and machining and modifying a micro-nano structure of the metal surface by combining a coupled bionic concept according to the correlation. A scientific basis and a novel design method are provided for the bionic regulation of the number of droplet bounces over the metal surface.

### CROSS-REFERENCE TO RELATED APPLICATIONS

This application claims priority to Chinese Patent Application No. 202410840471.7, filed on Jun. 26, 2024, which is hereby incorporated by reference in its entirety.

### TECHNICAL FIELD

The present invention relates to the technical field of metal surface wettability regulation, and specifically to a bionic regulation method for a number of bounces of a droplet on a metal surface based on acoustic in-situ monitoring.

### BACKGROUND

The common phenomena of icing and surface adhesion in nature have brought serious troubles to production and life, such as air crashes of civil airliners and military aircraft due to icing of the wings, and surface corrosion and reduced transportation efficiency of marine ships due to the adhesion of marine organisms.

The revelation of the whole-course contact behavior of the solid-liquid interface and the dynamic rebound mechanism of the droplet is a guide for solving the difficulties of anti-icing and anti-frost, self-cleaning and desorption on metal-based surfaces, and can also provide a scientific basis for the design of superhydrophobic and super-wettable bionic surfaces. For example, in “Interface Mechanical Behavior Mechanism and Performance Regulation of Bionic Superhydrophobic Surfaces”, Yang Konghua analyzed the spreading state and impact change process of a droplet on a bionic surface, and studied the asymmetric bouncing and directional transport of the droplet on the surface with wettability gradients.

However, the regulation of the number of droplet bounces over metal-based surfaces also has application value. For example, the probability that the droplet eventually stays on the surface can be greatly reduced by increasing the number of droplet bounces over metal-based surfaces in engineering, so that the ice accumulation on the surface is effectively inhibited. For another example, by reducing the number of droplet bounces over the metal-based heat exchange surface, Leidenfrost effect of a solid-liquid interface can be inhibited, and the contact between the droplet and the surface is promoted, thereby improving the surface cooling and heat exchange efficiency. However, in the prior art, due to the instantaneity, the fluidity and the adhesion of the solid-liquid interface contact process and the time-varying property and the complexity of the spreading and shrinking motion process of the droplet, the conventional testing technology represented by high-speed imaging is difficult to intuitively reveal the time-varying characteristics of energy dissipation such as pinning dissipation, viscous dissipation, adhesion dissipation in the solid-liquid interface and surface energy/kinetic energy conversion, which further limits the deep explanation of the bouncing behavior and the dynamic mechanism of the droplet, and thus is difficult to provide an effective scientific basis for the design of a metal-based multi-coupled bionic functional surface.

### SUMMARY

To solve at least one of the above problems, the present invention provides a bionic regulation method for a number of bounces of a droplet on a metal surface based on acoustic in-situ monitoring.

A technical solution of the present invention is as follows: a bionic regulation method for a number of bounces of a droplet on a metal surface based on acoustic in-situ monitoring comprises the following steps:
S1: testing metal superhydrophobic surfaces with different characteristics and different sizes based on an optical-acoustic synchronous in-situ testing system for droplet bounces to obtain dynamic contact characteristics and acoustic properties of the solid-liquid interface of the metal superhydrophobic surfaces with different characteristics and different sizes in the droplet bounce process; and
S2: obtaining a correlation among the metal superhydrophobic surface, the acoustic response and the droplet bounce behavior based on the dynamic contact characteristics and the acoustic properties of the solid-liquid interface, and machining and modifying a micro-nano structure of the metal surface by combining a coupled bionic concept according to the correlation.

Beneficial effects: according to the present invention, through the optical-acoustic coordinated in-situ monitoring of the solid-liquid contact interface, the dynamic behavior and the dynamics response of the whole continuous bouncing process of the droplet are synchronously obtained, the time-varying characteristic of interface adhesion and energy conversion can be studied and obtained, the correlation between the characteristics of the droplet pinning adhesion, the solid-liquid contact time and the like and the acoustic waveform characteristics is established, the correlation mapping between the acoustic response and a droplet bounce mechanism is formed, the influence mechanism and the law of a surface micro-nano structure and a Weber number on the droplet bounce behavior and the number of droplet rebounces are revealed, the bionic micro-nano structure design rule based on the droplet bounce characteristic regulation is created, a scientific basis and a novel design method are provided for the bionic regulation of the number of droplet bounces over the metal surface based on this design rule, and the regulation of the number of droplet bounces over the metal surface based on a requirement is facilitated. Particularly, the conventional testing technology represented by high-speed imaging is difficult to intuitively reveal the time-varying characteristics of energy dissipation such as pinning dissipation, viscous dissipation, adhesion dissipation in the solid-liquid interface and surface energy/kinetic energy conversion, which further limits the deep explanation of the bouncing behavior and the dynamic mechanism of the droplet, and thus is difficult to provide an effective scientific basis for the design of a metal-based multi-coupled bionic functional surface. The optical-acoustic coordinated in-situ monitoring for the droplet bounces in the present invention can identify the energy dissipation and surface energy/kinetic energy conversion of a solid-liquid interface by collecting the acoustic response of the droplet bounce, and can synchronously obtain the time-varying characteristics of the droplet motion and the acoustic response corresponding to each motion stage by high-speed imaging and acoustic emission nondestructive testing technology, so as to obtain the correlation among the metal superhydrophobic surface, the acoustic response and the droplet bounce behavior.

### BRIEF DESCRIPTION OF DRAWINGS

FIG. 1 is a schematic diagram of an optical-acoustic synchronous in-situ monitoring device for droplet bounces according to an embodiment;

FIG. 2 is a schematic diagram showing a continuous bouncing process of a droplet according to an embodiment;

FIG. 3 is a schematic diagram of pinning, residue-free and adhesion of a droplet on different surfaces according to an embodiment;

FIG. 4 is a waveform diagram collected by a broadband acoustic emission sensor and a spectrogram obtained by Fourier transform according to an embodiment;

FIG. 5 is a waveform diagram collected by a resonant acoustic emission sensor and a spectrogram obtained by Fourier transform according to an embodiment;

FIG. 6 is a spectrogram of impact of a droplet on an unmodified metal surface;

FIG. 7 is a spectrogram of impact of a droplet on a metal surface, which features a superhydrophobic microstructure with a spacing of 50 microns;

FIG. 8 is a spectrogram of impact of a droplet on a metal surface, which features a superhydrophobic microstructure with a spacing of 500 microns;

FIG. 9 is a spectrogram of impact of a droplet on a metal surface, which features a superhydrophobic microstructure with a spacing of 1000 microns;

FIG. 10 is a schematic diagram of a multi-level composite structure morphology of a metal surface; and

FIG. 11 is a graph showing comparison results between a predicted value and an actual value of a number of corresponding droplet bounces according to an embodiment.

In the drawings, 1: light source, 2: to-be-tested metal superhydrophobic surface, 3: droplet dripping device, 4: high-speed camera, 5: acoustic emission collecting device, 6: cone column, 7: microparticle, 8: hydrophobic layer, and 9: metal base.

### DETAILED DESCRIPTION OF THE EMBODIMENTS

The specific implementations of the present invention will be described clearly and completely below with reference to examples and drawings. It is clear that the described embodiments are merely a part rather than all of embodiments of the present invention.

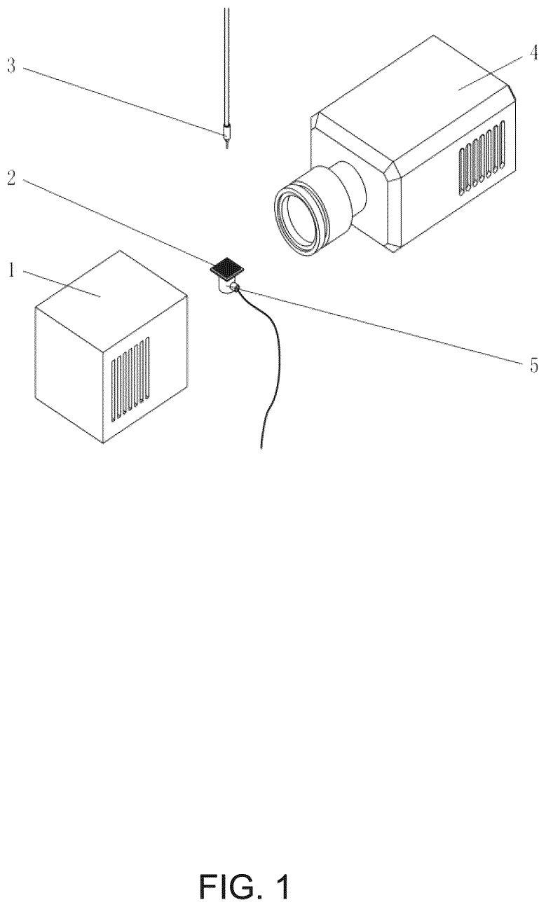

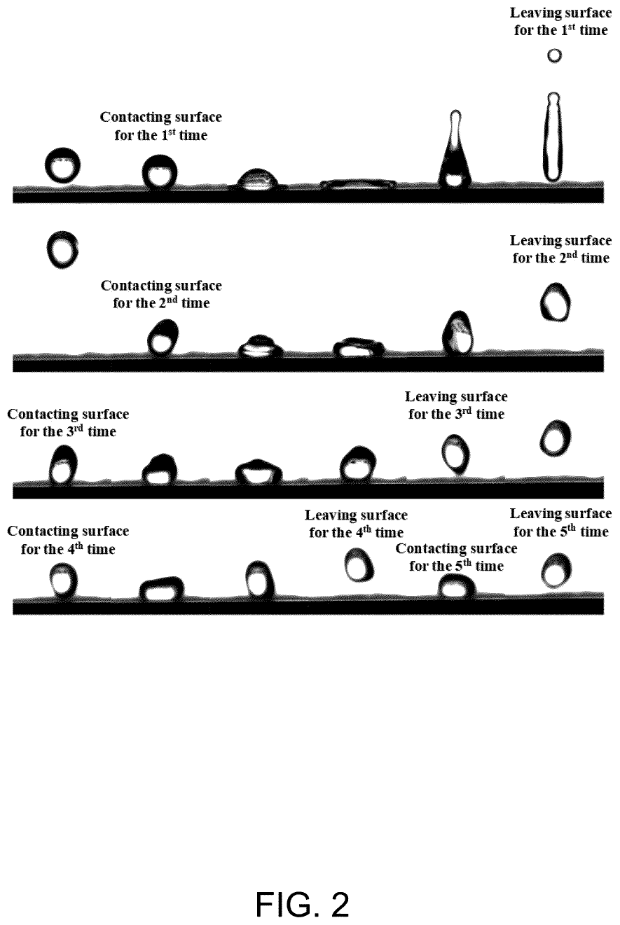

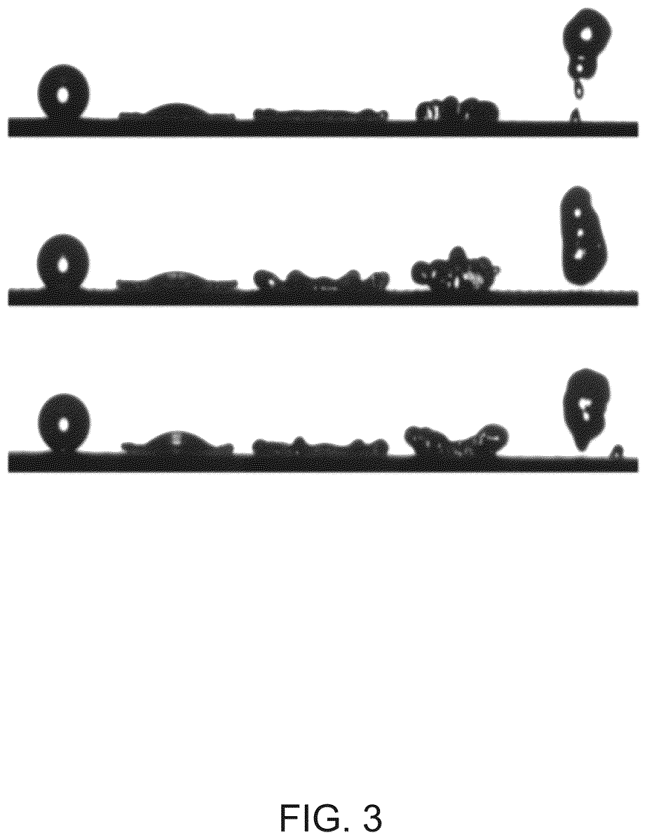

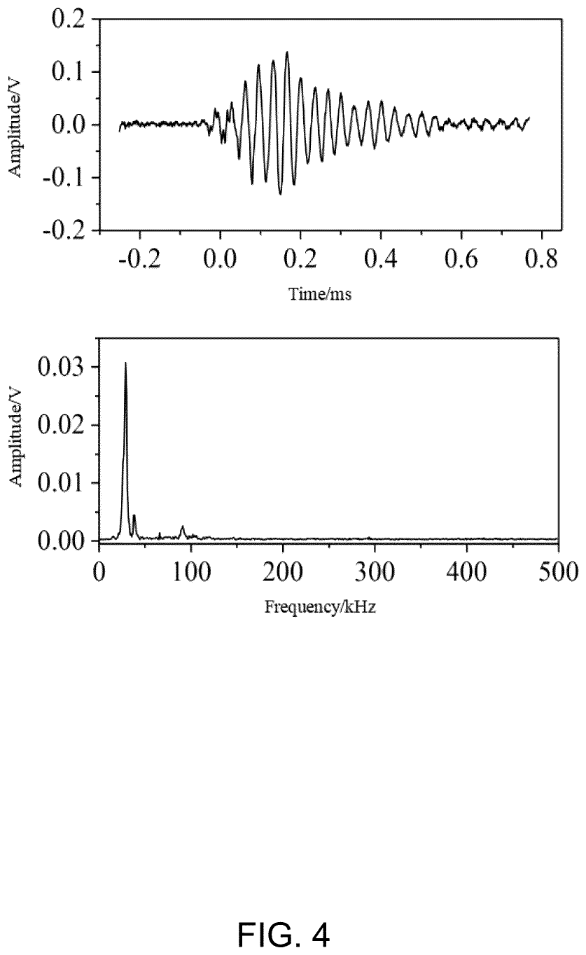

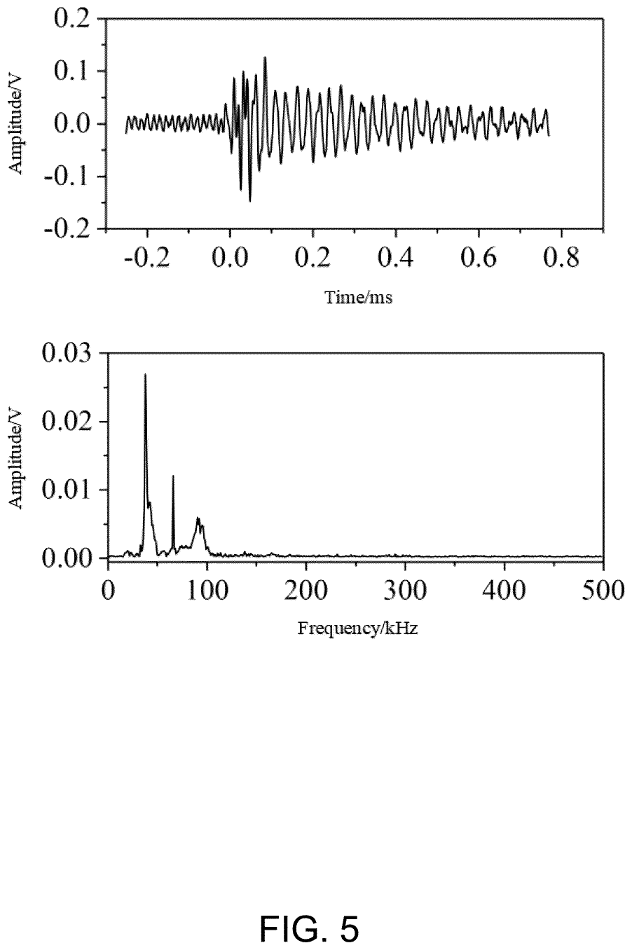

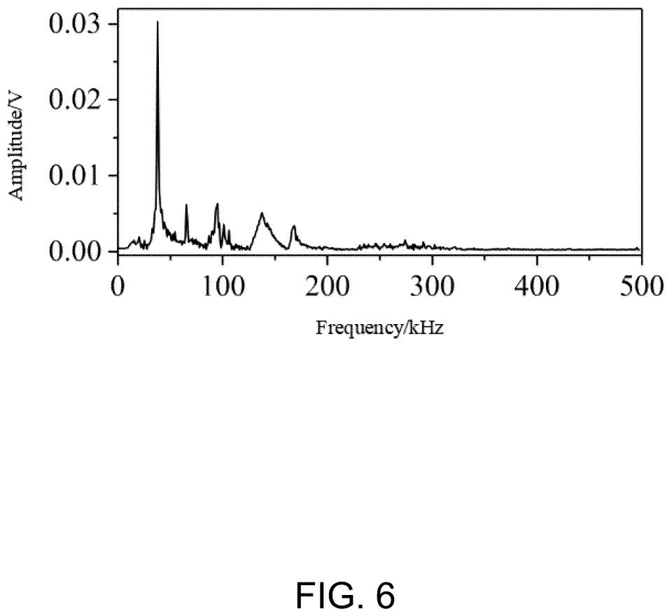

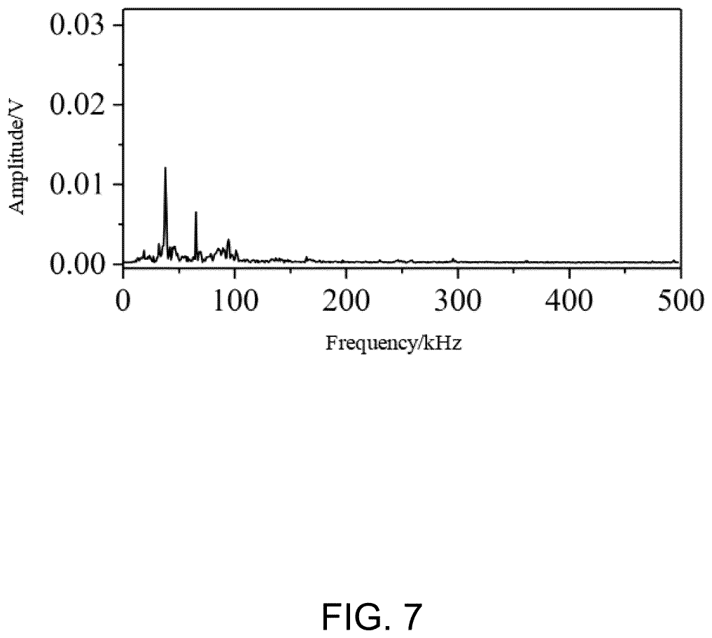

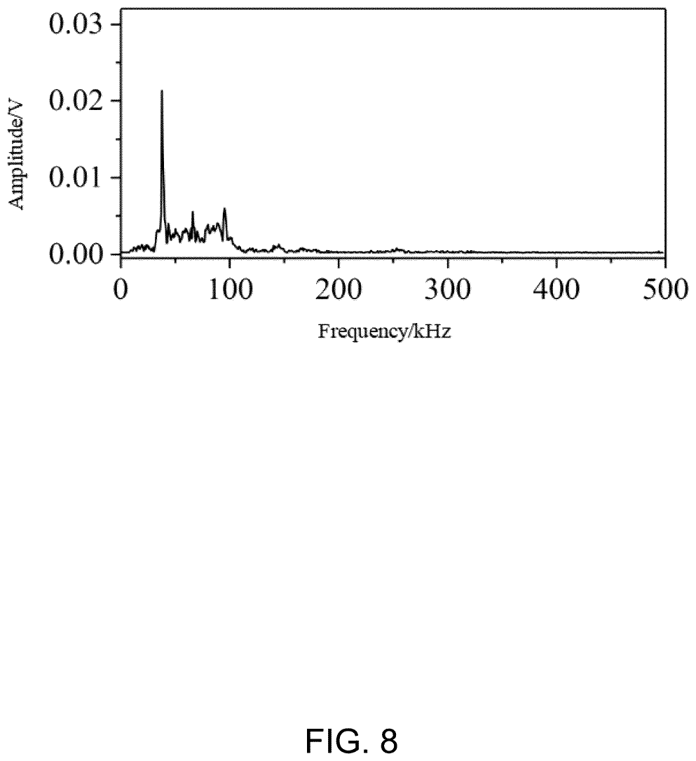

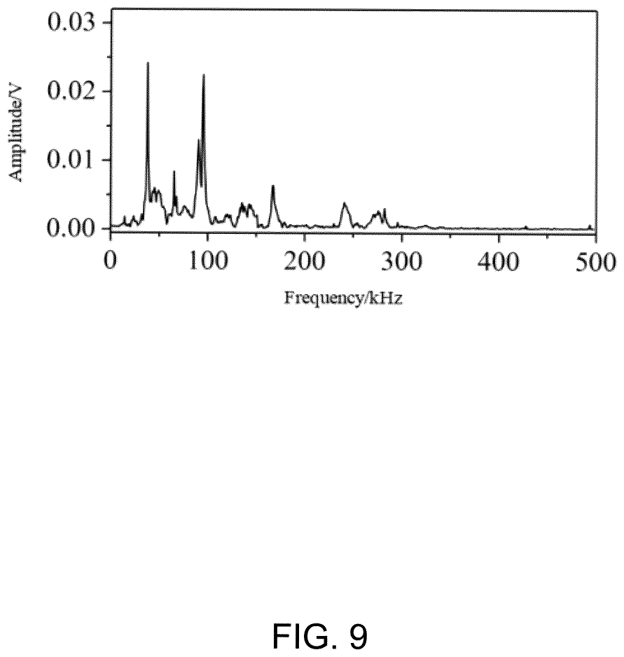

As shown in FIGS. 1 to 11, a bionic regulation method for a number of bounces of a droplet on a metal surface based on acoustic in-situ monitoring comprises the following steps:
S1: testing metal superhydrophobic surfaces with different characteristics and different sizes based on an optical-acoustic synchronous in-situ testing system for droplet bounces to obtain dynamic contact characteristics and acoustic properties of the solid-liquid interface of the metal superhydrophobic surfaces with different characteristics and different sizes in the droplet bounce process.

As shown in FIG. 1, in this step, the optical-acoustic synchronous in-situ testing system for droplet bounces is a device for testing the acoustic properties and the spreading and shrinking motion characteristics of the droplet when the droplet acts on the metal surface during the droplet bounce process. The testing system comprises a to-be-tested metal superhydrophobic surface, wherein at least one acoustic emission collecting device is adhered to a non-test surface of the to-be-tested metal superhydrophobic surface, a high-speed camera and a light source are respectively arranged on two sides of the to-be-tested metal superhydrophobic surface, a droplet dropping device is arranged right above the to-be-tested metal superhydrophobic surface, and a droplet dropped by the droplet dropping device drops on a to-be-tested surface of the to-be-tested metal superhydrophobic surface; and the acoustic emission collecting device comprises a broadband acoustic emission sensor and a resonant acoustic emission sensor. In the testing process, the high-speed camera can take a picture of actions such as spreading, shrinking and bouncing of the droplet on the metal surface at a high frame rate under the action of the light source so as to record the spreading and shrinking motion characteristics of the droplet; and the acoustic emission collecting device can detect the acoustic properties of the droplet when the droplet acts on the metal surface. Meanwhile, for the acoustic emission collecting device and the to-be-tested metal superhydrophobic surface, the acoustic emission collecting device can be directly adhered to the back of the to-be-tested metal superhydrophobic surface by coating a coupling agent on the to-be-tested metal superhydrophobic surface, and a plurality of acoustic emission collecting devices can be arranged on a lower portion of the to-be-tested metal superhydrophobic surface by arranging a placement table, which belong to conventional means in the field.

The specific metal superhydrophobic surface can be detected by the conventional means, such as the conventional scanning electron microscope and laser confocal microscope, which all belong to the conventional microscopic detection means, and therefore, the specific operations thereof are not described in detail.

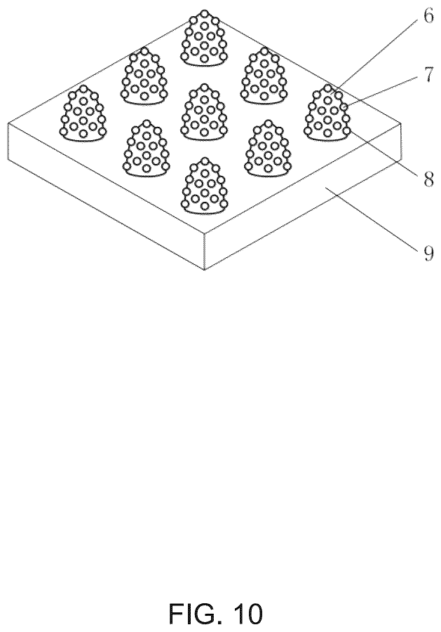

For the specific characteristics of the metal superhydrophobic surface, namely the superhydrophobic microstructure, the superhydrophobic microstructure comprises a cone column 6, a convex hull, a microparticle 7 and a multi-level composite structure morphology. The multi-level composite structure form is composed of a plurality of different types of micro-nano structures, as shown in FIG. 10, the cone column 6 is arranged on the surface of a metal base 9, the microparticle 7 is arranged on the surface of the cone column, a layer of hydrophobic layer 8 is further coated on the surfaces of the metal base 9, the cone column 6 and the microparticle 7, and the size of the superhydrophobic microstructure is also provided.

Meanwhile, the inventors also found that a distance between the superhydrophobic microstructures also has a great influence on the bouncing behavior of the droplet. For this reason, the inventors also add different distances between the superhydrophobic microstructures as one of the influencing factors of the bouncing behavior of the droplet in this embodiment.

The method for obtaining the dynamic contact characteristics and the acoustic properties of the solid-liquid interface comprises the following steps: after equipment is assembled, dripping a droplet on the to-be-tested metal superhydrophobic surface by using a droplet dripping device, obtaining acoustic information of the solid-liquid interface in the whole droplet bounce process in a wide frequency range by using the broadband acoustic emission sensor, identifying characteristic frequency of acoustic response of the solid-liquid interface during the droplet motion process, selecting the resonant acoustic emission sensor according to the characteristic frequency to obtain the acoustic information of the solid-liquid interface in a characteristic frequency range, obtaining acoustic signals of the solid-liquid interface in the whole process of continuous droplet bounce in a relatively higher sensitivity, processing the acoustic signals to obtain a waveform diagram and a spectrogram of the acoustic response of droplet bounce, and extracting the characteristic frequency of the acoustic response and the corresponding intensity; meanwhile, in the experimental process, the high-speed camera is used for photographing the dynamic characteristics of the spreading, shrinking and desorption processes of the droplet on the metal superhydrophobic surface.

Since the above operation requires measurement of acoustic characteristics, to avoid the influence of external factors on the test process, the test is usually performed under the condition that the environmental noise is less than 30 dB.

After the operation is performed, the dynamic contact characteristics and the acoustic properties of the solid-liquid interface of the metal superhydrophobic surfaces with different characteristics and sizes in the droplet bounce process can be obtained, which provides a large amount of data support for the subsequent steps.

Before experiments are performed, a large number of metal superhydrophobic surfaces with different characteristics and different sizes can be prepared by existing technologies, including wire electric discharge cutting, laser ablation, organic adsorption and the like, which belong to the conventional technical means in the field.

In this embodiment, a laser ablation machining technology is used, machining parameters such as laser power, scanning speed and scanning line spacing are adjusted, and organic adsorption surface modification is combined, so that metal superhydrophobic surfaces with different distribution spacings are prepared on the metal-based surface.

For a high-speed camera, the resolution of the high-speed camera is set to 1280×1024, the photographing frame rate is set to 10000 frames/second, the exposure time is set to 10 μs, and the storage length is set to 20000 frames.

For the acoustic emission collecting device, the collection threshold of the acoustic emission sensor is set to 30 dB, the preamplifier gain is set to 60 dB, a lower limit and an upper limit of an analog filter are respectively set to 1 kHz and 3 MHz, a sampling rate is set to 5 MSPS, the data storage length is set to 15 k, and a test end face of the acoustic emission sensor is connected to a test piece by a coupling agent.

The WSα acoustic emission sensor is selected as a broadband acoustic emission sensor with a peak sensitivity of 55 dB, an operating frequency range of 100-1000 kHz, and a resonant frequency of 125 kHz.

The R6α acoustic emission sensor is selected as a resonant acoustic emission sensor with a peak sensitivity of 75 dB, an operating frequency range of 35-100 kHz, and a resonant frequency of 55 kHz.
S2: obtaining a correlation among the metal superhydrophobic surface, the acoustic response and the droplet bounce behavior based on the dynamic contact characteristics and the acoustic properties of the solid-liquid interface, and machining and modifying a micro-nano structure of the metal surface by combining a coupled bionic concept according to the correlation.

The method for obtaining the correlation among the metal superhydrophobic surface, the acoustic response and the droplet bounce behavior comprises the following steps: based on the dynamic contact characteristics and the acoustic properties of a solid-liquid interface, obtaining the correlation among the structure appearance, the size and the number of droplet bounces by combining the dynamic behavior of the droplet obtained by high-speed imaging in the spreading, shrinking and desorption processes of different metal superhydrophobic surfaces; analyzing the time-varying law of transient acoustic response generated in the droplet bounce process to obtain an acoustic response spectrogram and obtain the influence of the structure appearance and the size on the pinning adhesion of the solid-liquid interface and the energy dissipation of the droplet; and thereby obtaining the correlation among the metal superhydrophobic surface, the acoustic response and the droplet bounce behavior.

After the correlation among the metal superhydrophobic surface, the acoustic response and the droplet bounce behavior is obtained, this can explain the following: the respective effects of the optical high-speed imaging and the acoustic characteristics, the energy dissipation of the droplet corresponding to the acoustic characteristics (including the forms of pinning, adhesion, friction and the like of a solid-liquid interface, the energy dissipation can directly influence the number of droplet bounces, and a high-speed camera cannot fully identify the energy dissipation because the motion process involving fluids and interfaces is difficult to see and the energy dissipation, conversion, and transmission are invisible to the naked eye and camera), the number of droplet bounces corresponding to the dynamic process/the droplet bounce behavior photographed by the high-speed camera, and the partially visible pinning adhesion phenomenon related to the energy dissipation.

In this embodiment, the correlation between the superhydrophobic structure and the acoustic information is analyzed, and it is found that the superhydrophobic surface with the plane and the large structure spacing has significant acoustic response in the frequency range of 120-300 kHz, and the characteristic peak amplitude at 38 kHz is significantly higher and corresponds to the adhesion of a solid-liquid interface. For small and medium structure spacings with good superhydrophobicity, there is no significant acoustic response in the frequency range of 120-300 kHz, and the characteristic peak amplitude at 38 kHz is relatively low, and the surface adhesion effect is weak.

In this embodiment, the correlation between the acoustic information and the number of droplet bounces is analyzed, and it is found that the number of droplet bounces is significantly influenced by energy dissipation caused by a pinning effect and an adhesion effect in the droplet bounce process.

The pinning effect can be distinguished by the ratios of the characteristic peak amplitudes at 66 kHz and 96 kHz (the characteristic peak amplitude at 66 kHz divided by the characteristic peak amplitude at 96 kHz). The ratios of the characteristic peak amplitudes at 66 kHz and 96 kHz for the bouncing of the droplet on the superhydrophobic surface with the structure spacings of 50 μm, 500 μm and 1000 μm are respectively 2.133, 0.915 and 0.371, indicating that the pinning effect of the solid-liquid interface gradually weakens with the increase of the structure spacing, which is beneficial to increasing the number of droplet bounces.

The adhesion effect can be distinguished by the characteristic peak amplitude at 38 kHz. The characteristic peak amplitudes of 38 kHz acoustic response for the bouncing of the droplet on the superhydrophobic surface with the structure spacings of 50 μm, 500 μm and 1000 μm are respectively 0.0120 V, 0.0213 V and 0.0241 V, indicating that the adhesion effect of the solid-liquid interface gradually enhances with the increase of the structure spacing, and too large structure spacing is not beneficial to the improvement of the number of droplet bounces.

In this embodiment, after analyzing the correlation between the structure spacing and the number of droplet bounces, it is found that there is a clear correlation between the structure spacing and the number of droplet bounces, which can be represented by the following formula:

$$\{ \begin{matrix}R = N - \frac{A}{S} & S < {S}_{0} \\ R = N - \frac{A}{S} - {B ( S - {S}_{0} )}^{2} & S \geq {S}_{0}\end{matrix}$$

wherein R is the number of droplet bounces, S is a structural distance, S0 is a critical structure spacing of the adhesion residue of the droplet, which is equal to the minimum structure spacing of the adhesion residue of the droplet, N, A and B are correction constants, and the pinning and the adhesion of the solid-liquid interface are regulated by changing the structure spacing, so as to change the bounce behavior and the energy dissipation of the droplet and realize the regulation of the number of bounces of the droplet on the metal surface based on a requirement.

In this embodiment, taking the initial conditions of droplet impact with a droplet falling height of 40 mm and a droplet diameter of 2.05 mm (equivalent Weber number of 25.2) as an example, the critical structure spacing of the adhesion residue of the droplet is 500 μm, and the averages of 5 experiments for the number of bounces of the droplet on the superhydrophobic surface with the structure spacings of 50 μm, 500 μm and 1000 μm are respectively 4, 11.2 and 3.2, and the to-be-determined constants N, A and B are calculated by substituting the averages into a formula, which are respectively equal to 12, 400 and 3.4×10⁻⁵. For this initial condition, a correlation formula of the number of droplet bounces and the structure spacing can be represented as:

$$\{ \begin{matrix}R = 12 - \frac{400}{S} & S < 500 \\ R = 12 - \frac{400}{S} - 3.4 \times {10}^{- 5} {( S - 500 )}^{2} & S \geq 500\end{matrix}$$

For this initial condition, the structure spacing can be selected based on a requirement according to a correlation formula of the number of droplet bounces and the structure spacing, so that the target number of droplet bounces is achieved.

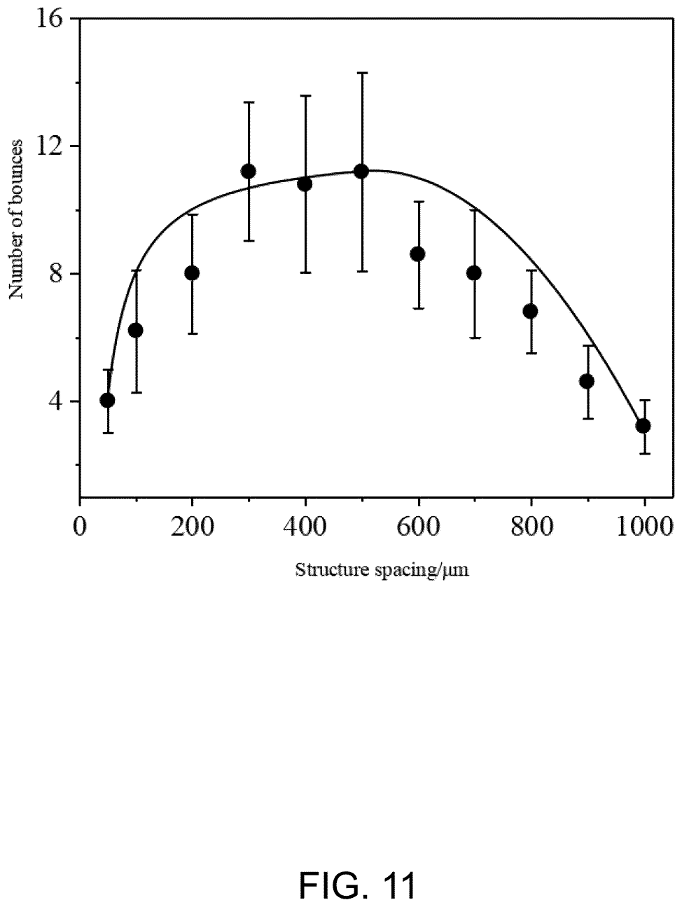

The final predicted and controlled number of bounces and the actual number of bounces are shown in FIG. 11. In FIG. 11, the curve represents the predicted and controlled number of bounces, and the scattered points represent the actual number of bounces. It can be seen from FIG. 11 that the predicted result of this embodiment is close to the actual number of bounces, indicating that the result of this embodiment is more accurate.

The above descriptions are only preferred embodiments of the present invention, and are not intended to limit the present invention in any form. Although the preferred embodiments above have disclosed the present invention, they are not intended to limit the present invention. Any of those familiar with the technical field, without departing from the scope of the technical solutions of the present invention, can use the technical content disclosed above to make various changes and modify the technical content as equivalent changes of the equivalent embodiments. However, any simple modifications, equivalent changes and modifications made to the above embodiments according to the technical spirit of the present invention without departing from the content of the technical solutions of the present invention shall fall within the scope of the technical solutions of the present invention.

What is claimed is:

1. A bionic regulation method for a number of bounces of a droplet on a metal surface based on acoustic in-situ monitoring, comprising:
S1: testing metal superhydrophobic surfaces with different surface information based on an optical-acoustic synchronous in-situ testing system for droplet bounces to obtain dynamic contact characteristics and acoustic properties of the solid-liquid interface of the metal superhydrophobic surfaces with different surface information in the droplet bounce process; and
S2: obtaining a correlation among the metal superhydrophobic surface, the acoustic response and the droplet bounce behavior based on the dynamic contact characteristics and the acoustic properties of the solid-liquid interface, and machining and modifying a micro-nano structure of the metal surface by combining a coupled bionic concept according to the correlation.

2. The method according to claim 1, wherein the surface information of the metal superhydrophobic surface comprises: superhydrophobic microstructures with different forms on the metal superhydrophobic surface and different distances between the superhydrophobic microstructures.

3. The method according to claim 2, wherein the superhydrophobic microstructure comprises a cone column, a convex hull, a microparticle and a multi-level composite structure morphology.

4. The method according to claim 2, wherein the surface information of the metal superhydrophobic surface is obtained using equipment comprising a scanning electron microscope and a laser confocal microscope.

5. The method according to claim 1, wherein the optical-acoustic synchronous in-situ testing system for droplet bounces comprises a to-be-tested metal superhydrophobic surface, at least one acoustic emission collecting device is adhered to a non-test surface of the to-be-tested metal superhydrophobic surface, a high-speed camera and a light source are respectively arranged on two sides of the to-be-tested metal superhydrophobic surface, a droplet dropping device is arranged right above the to-be-tested metal superhydrophobic surface, and a droplet dropped by the droplet dropping device drops on a to-be-tested surface of the to-be-tested metal superhydrophobic surface; and the acoustic emission collecting device comprises a broadband acoustic emission sensor and a resonant acoustic emission sensor.

6. The method according to claim 5, wherein the method for obtaining the dynamic contact characteristics and the acoustic properties of the solid-liquid interface comprises the following steps: after equipment is assembled, dripping a droplet on the to-be-tested metal superhydrophobic surface by using the droplet dripping device, obtaining acoustic information of the solid-liquid interface in the whole droplet bounce process in a wide frequency range by using the broadband acoustic emission sensor, identifying characteristic frequency of acoustic response of the solid-liquid interface during the droplet motion process, selecting the resonant acoustic emission sensor according to the characteristic frequency to obtain the acoustic information of the solid-liquid interface in a characteristic frequency range, obtaining acoustic signals of the solid-liquid interface in the whole process of continuous droplet bounce in a relatively higher sensitivity, processing the acoustic signals to obtain a waveform diagram and a spectrogram of the acoustic response of droplet bounce, and extracting the characteristic frequency of the acoustic response and the corresponding intensity; meanwhile, in the experimental process, the high-speed camera is used for photographing the dynamic characteristics of the spreading, shrinking and desorption processes of the droplet on the to-be-tested surface of the metal superhydrophobic surface.

7. The method according to claim 1, wherein the method for obtaining a correlation among the metal superhydrophobic surface, the acoustic response and the droplet bounce behavior comprises the following steps: based on the dynamic contact characteristics and the acoustic properties of the solid-liquid interface, obtaining a correlation among the structure appearance, the size and the number of droplet bounces by combining the dynamic behavior of the droplet obtained by high-speed imaging in the spreading, shrinking and desorption processes of different metal superhydrophobic surfaces; analyzing a time-varying law of transient acoustic response generated in the droplet bounce process to obtain an acoustic response spectrogram and obtain the influence of the structure appearance and the size on the pinning adhesion of the solid-liquid interface and the energy dissipation of the droplet; and thereby obtaining the correlation among the metal superhydrophobic surface, the acoustic response and the droplet bounce behavior.

8. The method according to claim 1, wherein the machining and modifying a micro-nano structure of the metal surface comprises the following steps: based on the correlation among the metal superhydrophobic surface, the acoustic response and the droplet bounce behavior, by combining a coupled bionic concept of a non-smooth morphology, a multi-level composite structure and a water-repellent coating and taking restraining a pinning effect, retarding interface adhesion and improving the number of bounces as an objective, preparing the metal superhydrophobic surface with a required number of bounces by machining and modifying the metal surface by utilizing modification technologies comprising wire electric discharge cutting, laser ablation and organic adsorption.
